(19) United States
(12) Patent Application Publication
(10) Pub. No.: US 2026/0284091 A1
(43) Pub. Date: Sep. 24, 2026

(54) IMIDAZOLE LOADED GLYCYRRHIZIC ACID HYDROGELS

(71) Applicant: KATHOLIEKE UNIVERSITEIT LEUVEN, Leuven (BE)

(72) Inventors: Bruno CAMMUE, Alsemberg (BE); Maarten MEES, Horebeke (BE); Hans STEENACKERS, Leuven (BE); Karin THEVISSEN, Bierbeek (BE); Wilm THIELEMANS, Kortrijk (BE); Thijs VACKIER, Dendermonde (BE)

(21) Appl. No.: 19/474,812

(22) PCT Filed: Apr. 15, 2024
(86) PCT No.: PCT/EP2024/060183

§ 371 Date: Oct. 13, 2025

(30) Foreign Application Priority Data
Apr. 14, 2023 (EP) 23168100.8

(51) Int. Cl. A61K 31/704; A61K 9/06; A61K 31/4174; A61K 31/496; A61P 31/04; A61P 31/10

(52) U.S. Cl. CPC A61K 31/704; A61K 9/06; A61K 31/4174; A61K 31/496; A61P 31/04; A61P 31/10

(57) ABSTRACT

The present application relates to a glycyrrhizic acid hydrogel with a pH of between 2.5 and 5.0 and comprising an imidazole, for use in the treatment or prevention of a bacterial or fungal infection. The application further relates to a pharmaceutical composition of said hydrogel and a method of treating or preventing a bacterial or fungal infection in an individual comprising the step of administering an effective aammoouunntt of an imidazole in aa glycyrrhizic acid hydrogel, a pharmaceutically acceptable salt or solvate thereof, with a pH of between 2.5 and 5.0.

### FIELD OF THE INVENTION

The present invention relates to hydrogel compositions comprising an imidazole in a glycyrrhizic acid or analogues thereof, within a specific acidic pH range, and the use of such compositions in the treatment or prevention of bacterial and fungal infections and for use as a coating to prevent infections on medical devices.

### BACKGROUND OF THE INVENTION

Different therapeutic properties of glycyrrhizic acid have been reported: anti-viral, anti-inflammatory, antitumoral and hepatoprotective [Azaz & Segal (1980) Pharm Acta Helv. 55, 183-186]. Due to the amphiphilic nature of glycyrrhizic acid, aggregates are formed in aqueous solution eventually leading to gel formation. The formation of a hydrogel of glycyrrhizic acid is related to its acidity and the concentration of glycyrrhizic acid.

Different studies are available on the antibacterial potential of glycyrrhizic acid in solution—not as a hydrogel—with reported activities against: Staphylococci, Enterococci, Helicobacter pylori, Bacillus subtitilis and Pseudomonas aeruginosa. In contrast, several studies also reported the lack of activity of glycyrrhizic acid in solution against Escherichia coli, Proteus vulgaris, Candida albicans and Streptococci. Zhao et al. (2020) ACS Appl Bio Mater 3, 648-653] evaluated the antibacterial activity of a 0.13 wt % glycyrrhizic acid hydrogel in PBS with reported activity against Staphylococcus aureus but with no effect against Escherichia coli.

US 2021/0015965 discloses glycyrrhizic acid hydrogels for wound healing.

Imidazoles like miconazole are characterized by antifungal and antibacterial activity. The current imidazole-containing hydrophobic creams for local administration, e.g. to treat vulvovaginal candidiasis (VVC), lack control over drug delivery as they often have unwanted leakage and short tissue exposure, resulting in limited killing of the pathogen. This leads to the re-occurrence of infections or even to drug resistance [Teixeira et al. (2023) Eur J Pharm Sci. 26, 188]. In case of vulvovaginal candidiasis, recurrence occurs due to the reemergence of Candida hyphae from the deep layers of the vaginal tissue [Melo et al. (2020) Carbohydr Polym. 230, 115608]. In general, shortly after finishing the treatment, approximately half of all women experience recurrent vulvovaginal candidiasis.

To overcome the limitations of the current pharmacological therapies, based on the conventional pharmaceutical dosage forms in hydrophobic creams, and ensure effective killing of the pathogen, treatments need to be developed that are compatible with the aqueous environment, biocompatible, have high porosity, and can deliver drugs in a controlled manner [Gosecka & Gosecki (2021) Pharmaceutics 13, 1393]. 575468; PMCID: PMC8469626]. Hydrogels are ideal candidates for various applications as they have tunable mechanical and flow properties ensuring a longer exposure time compared to current systems. Hydrogels can be specifically tuned to mimic the vaginal environment in terms of pH and moisture level.

### SUMMARY OF THE INVENTION

The present invention discloses methods of increasing the antibacterial/bactericidal activity and the activity spectrum of glycyrrhizic acid hydrogels, by decreasing the pH of the gel below pH 5.0, more specifically between pH 3.0 and pH 4.85. Addition of an imidazole further increases the antibacterial activity and even provides an antifungal activity

The basal antibacterial/bactericidal activity of the glycyrrhizic acid hydrogels on themselves cannot be mimicked by reducing the pH of the growth medium or a comparable concentration of glycyrrhizic acid in solution, that is to say not a hydrogel. Hence, it is the combination of the glycyrrhizic acid hydrogel (and not glycyrrhizic acid in solution) at a certain pH that results in the unexpected broad antibacterial/bactericidal activity of these hydrogels.

The present invention demonstrates that decreasing the pH of the hydrogel increases the antibacterial activity against bacterial strains against which glycyrrhizic acid in solution (at pH 7) is not active. Decreasing the pH of the glycyrrhizic acid hydrogel to pH 4.2 already increases the antibacterial activity against Acinetobacter baumannii, Methicillin-resistant Staphylococcus aureus (MRSA), Staphylococcus epidermidis, Streptococcus pyogenes and Enterococcus faecalis. Further decreasing the pH of the glycyrrhizic acid hydrogel to pH 3.8 broadens the activity spectrum to include Pseudomonas aeruginosa. Finally, decreasing the pH of the glycyrrhizic acid hydrogel even further to pH 3 broadens the activity spectrum of the hydrogel to include Escherichia coli, Klebsiella aerogenes and Klebsiella pneumoniae.

Further inclusion of an imidazole increases the antibacterial activity and also provides an antifungal activity.

Miconazole, an antifungal drug with fungicidal activity, was formulated in glycyrrhizic acid-based hydro- and organo-gels. Fungicidal and bactericidal activity of the creams and hydro/organo-gels was assessed using an in vitro activity test in which the microbial cultures are brought into contact with the creams and hydro/organo-gels for 1-2 h and remaining viable cells are quantified upon plating. Miconazole-glycyrrhizic acid hydrogels and miconazole-glycyrrhizic acid organogels were superior in in vitro fungicidal and bactericidal activity against Candida albicans, Candida glabrata, Staphylococcus aureus, as compared to commercial cream with miconazole (Daktarin™).

The invention is further summarised in the following statements.
1. A glycyrrhizic acid hydrogel comprising an imidazole or salt or solvate thereof for use in the prevention or treatment of a bacterial or fungal infection.
2. The hydrogel according to statement 1, for use in the prevention or treatment of a bacterial or fungal infection, wherein the hydrogel has a pH of between 2.5 and 5.
3. The hydrogel according to statement 1 or 2, for use in the prevention or treatment of a bacterial or fungal infection, wherein the hydrogel has a pH of between 3.5 and 4.5.
4. The hydrogel according to any one of statements 1 to 3, for use in the prevention or treatment of a bacterial or fungal infection, wherein the hydrogel has a pH of 4.
5. The hydrogel according to any one of statements 1 to 4, for use in the prevention or treatment of a bacterial or fungal infection, wherein the imidazole is miconazole, ketonazole or clotrimazole.
6. The hydrogel according to any one of statements 1 to 5, for use in the prevention or treatment of a bacterial or fungal infection, wherein the imidazole is miconazole.
7. The hydrogel according to any one of statements 1 to 6, for use in the prevention or treatment of a bacterial or fungal infection, wherein the concentration of the imidazole in the hydrogel is between 1.5 and 2.5 wt %.
8. The hydrogel according to any one of statements 1 to 7, for use in the prevention or treatment of a bacterial or fungal infection, wherein the concentration of glycyrrhizic acid is between 2.5 and 7.5 wt. %.
9. The hydrogel according to any one of statements 1 to 8, for use in the prevention or treatment of a bacterial or fungal infection, wherein the concentration of glycyrrhizic acid is between 4 and 6 wt. %.
10. The hydrogel according to any one of statements 1 to 9, for use in the prevention or treatment of a bacterial infection.
11. The hydrogel according to any one of statements 1 to 10, for use in the prevention or treatment of a bacterial infection, wherein the bacterial infection is an infection of Staphylococcus aureus or of Gardnerella vaginalis.
12. The hydrogel according to any one of statements 1 to 9, for use in the prevention or treatment of a fungal infection.
13. The hydrogel according to any one of statement 1 to 9, or to statement 12, for use in the prevention or treatment of a fungal infection, wherein the fungal infection is of Candida albicans or Candida glabrata.
14. A glycyrrhizic acid hydrogel characterized in that the hydrogel comprises an imidazole or salt or solvate thereof.
15. The hydrogel according to statement 14, wherein the hydrogel has a pH of between 2.5 and 5.
6. The hydrogel according to statement 14 or 15, wherein the hydrogel has a pH of between 3.5 and 4.5.
17. The hydrogel according to statement any one of statements 14 to 16, wherein the hydrogel has a pH of 4.
18. The hydrogel according to any one of statements 14 to 17, wherein the imidazole is miconazole, ketonazole or clotrimazole.
19. The hydrogel according to any one of statements 14 to 18, wherein the imidazole is miconazole.
20. The hydrogel according to any one of statements 14 to 19, wherein the concentration of the imidazole in the hydrogel is between 1.5 and 2.5 wt %.
21. The hydrogel according to any one of statement 14 to 20, wherein the concentration of the imidazole in the hydrogel is between 1.5 and 2.5 wt %.
22. The hydrogel according to any one of statements 14 to 21, wherein the concentration of glycyrrhizic acid is between 2.5 and 7.5 wt. %.
23. The hydrogel according to any one of statements 14 to 22, wherein the concentration of glycyrrhizic acid is between 4 and 6 wt. %.
24. The hydrogel according to any one of statements 14 to 23, wherein the concentration of glycyrrhizic acid is between 4 and 6 wt. %.
25. The hydrogel according to any one of statements 14 to 24, for use as a medicament.
26. A method of treating a bacterial or fungal infection comprising the step of administering an effective amount of hydrogel according to any one of statements 14 to 24.
7. A method of formulating an imidazole or salt or solvate thereof in a glycyrrhizic acid hydrogel, the method comprising the steps of: a) preparing an aqueous solution of an imidazole,
b) heating the solution of a),
c) optionally adjusting the pH of the solution of a) or adjusting the pH of the heated solution of b) in order to solubilize the imidazole,
d) adding glycyrrhizic acid to the heated solution of b),
e) adjusting the pH of the solution of d) to a pH between 2.5 and 5,
f) cooling the solution of d) and allowing the formation of a hydrogel.
28. The method according to statement 27, wherein the imidazole is added in an amount to obtain a 2 wt. % concentration in the hydrogel.
29. The method according to statement 27 or 38, wherein the glycyrrhizic acid is added in an amount to obtain a 5 wt. % concentration in the hydrogel.
30. The method according to any one of statements 27 to 29, wherein in step a) or b) the imidazole is miconazole and the pH is adjusted to pH 7.
31. The method according to any one of statements 27 to 30, wherein in step a) or b) the imidazole is clotrimazole and the pH is adjusted to pH 2.
32. The method according to any one of statements 27 to 31, wherein in step e), the pH is adjusted to a pH between 3.5 and 4.5.
33. The method according to any one of statements 27 to 31, wherein in step e), the pH is adjusted to a pH of 4.

### DETAILED DESCRIPTION

Figure legends

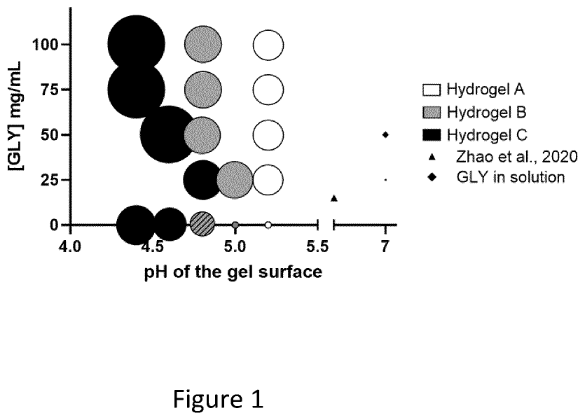

FIG. 1 Decreasing the pH of the GLY-hydrogel increases the activity spectrum.

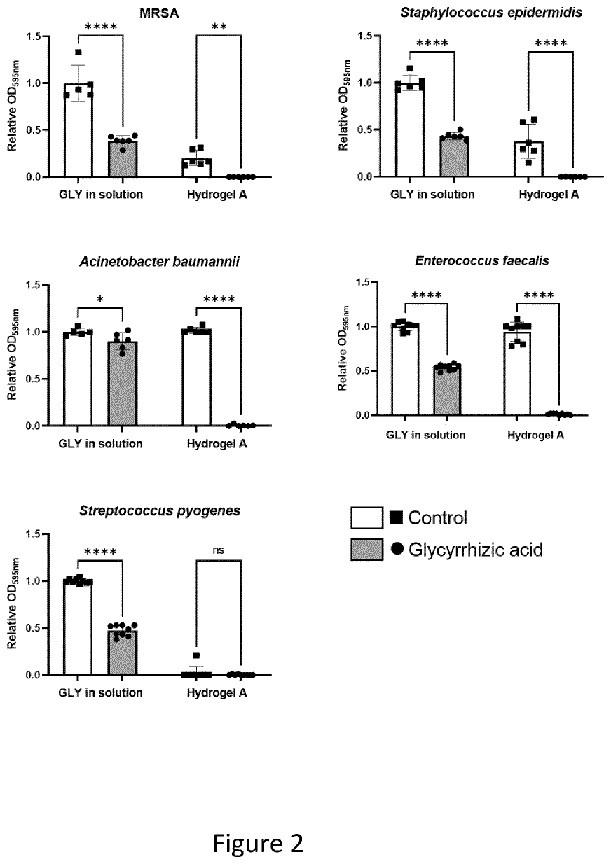

FIG. 2 Growth inhibiting activity of Hydrogel A

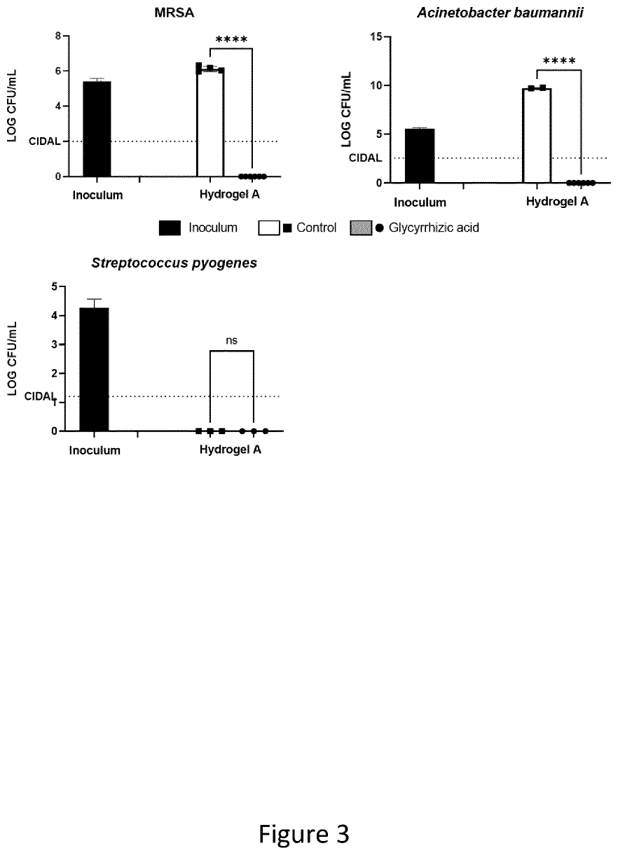

FIG. 3 Bactericidal activity of Hydrogel A

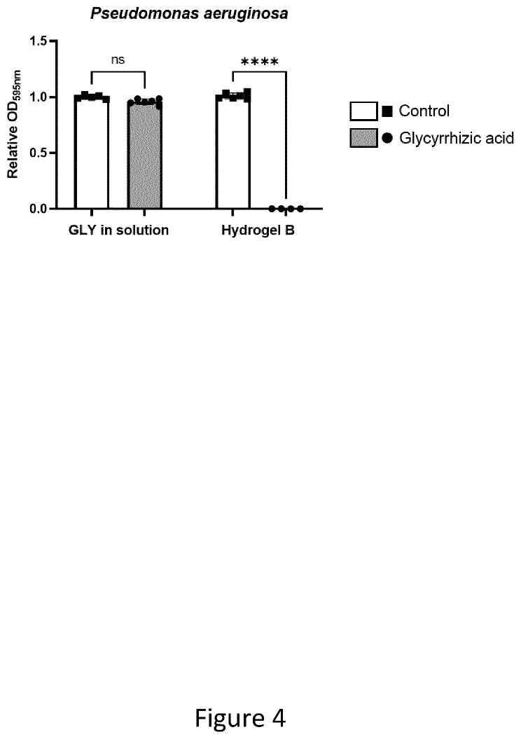

FIG. 4 Growth inhibiting activity of hydrogel B

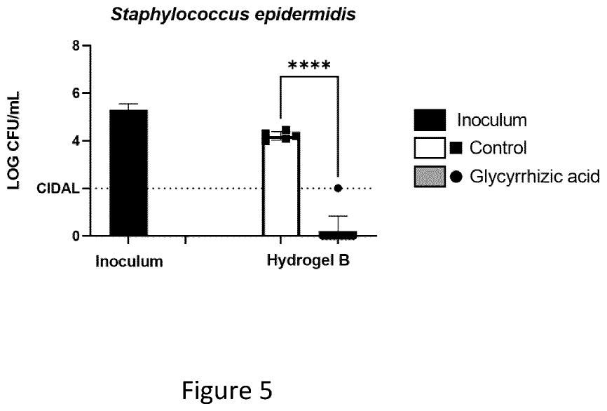

FIG. 5 Bactericidal activity of Hydrogel B

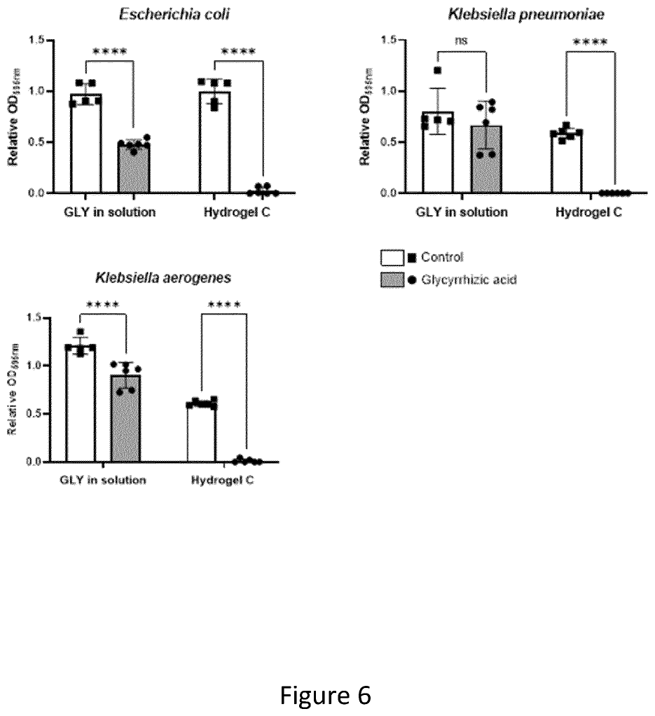

FIG. 6 Growth inhibiting activity of Hydrogel C

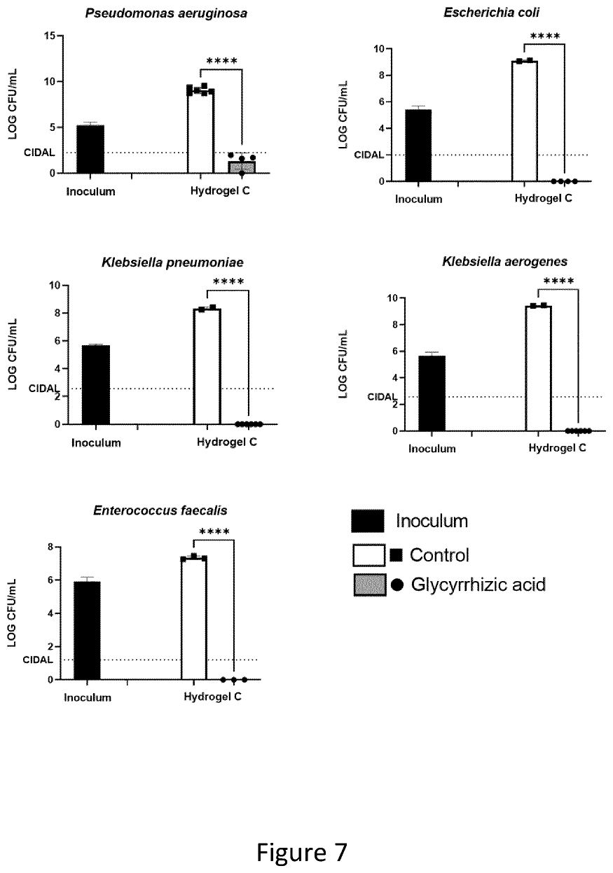

FIG. 7 Bactericidal activity of Hydrogel C

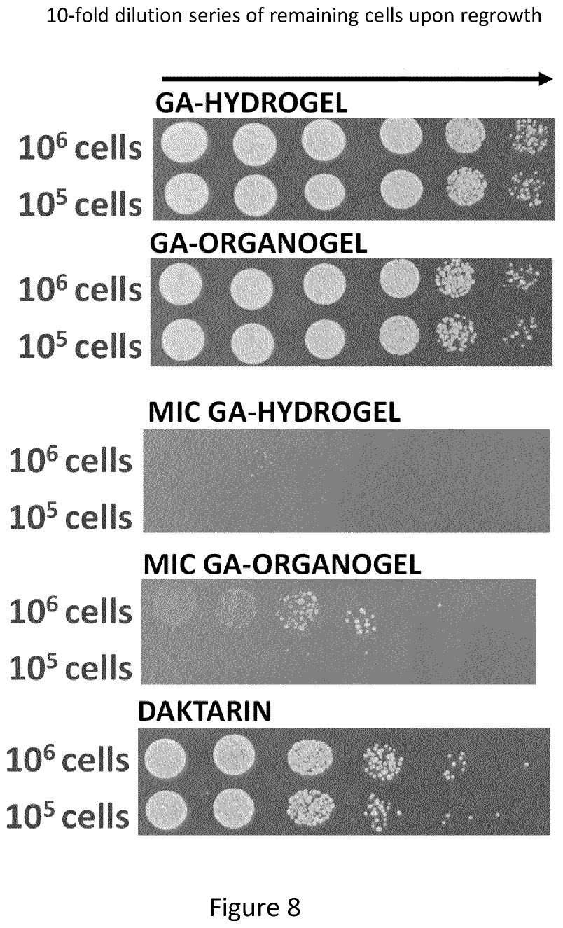

FIG. 8 spot assay of antifungal activity of unloaded and loaded hydrogels and organogels using 105 or 106 C. albicans cells. Filter show from left to right, a 10 fold dilution of cells. Daktarin™ is a commercial miconazole nitrate crème.

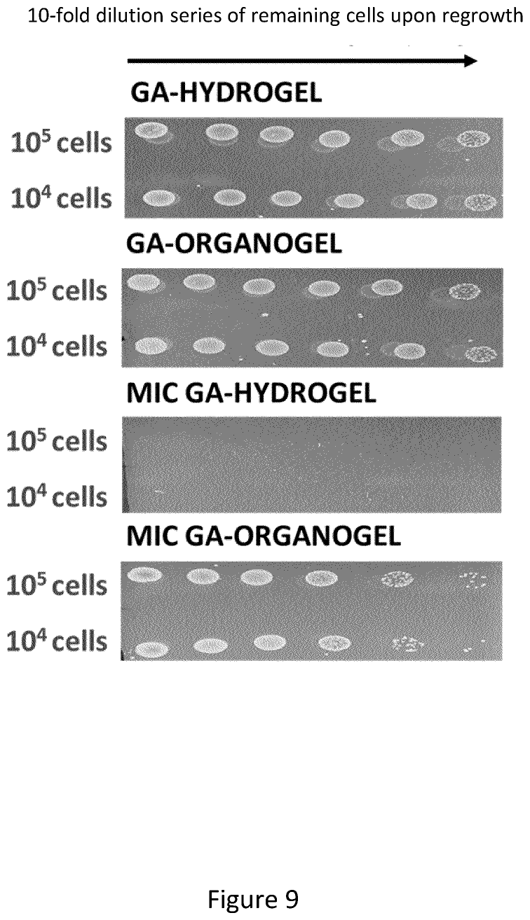

FIG. 9 spot assay of antibacterial activity of unloaded and loaded hydrogels and organogels using 104, 105 or 106 S. aureus cells. Filter show from left to right, a 10 fold dilution of cells.

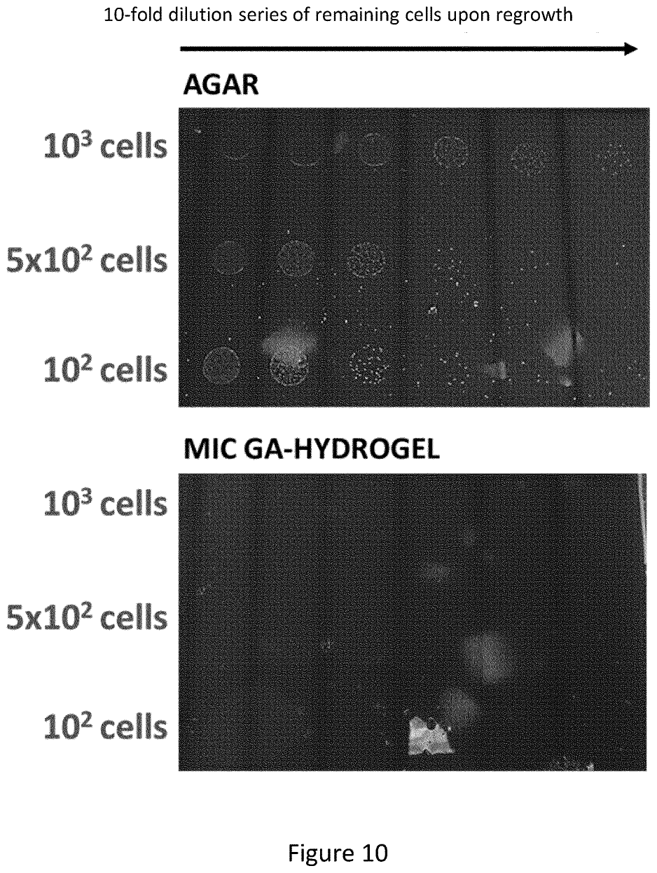

FIG. 10 spot assay of antifungal activity of an unloaded agar gel and a loaded glycyrrhizic acid hydrogel using 1000, 500 and 100 Gardnerella vaginalis cells. Filter show from left to right, a 10 fold dilution of cells.

Abbreviations in the figures are: GLY or GA: glycyrrhizic acid; Hydrogel A: a glycyrrhizic acid hydrogel with a gel pH of 4,2. This hydrogel acidifies the growth medium above the hydrogel (gel surface) to a pH of 5,2.; Hydrogel B: a glycyrrhizic acid hydrogel with a gel pH of 4,2. This hydrogel acidifies the growth medium above the hydrogel (gel surface) to a pH of 4.8-5.0 depending on the percentage of glycyrrhizic acid in the hydrogel and the amount of this gel used in the assay; Hydrogel C: a glycyrrhizic acid hydrogel with a gel pH of 3.0. This hydrogel acidifies the growth medium above the hydrogel (gel surface) to a pH of 4.4-4.8 depending on the percentage of glycyrrhizic acid in the hydrogel and the amount of this gel used in the assay.

MIC: miconazole; KET: ketonazole; CLO: Clotrimazole.

Hydrogel: According to the IUPAC gold Book, a hydrogel is defined as a gel in which the swelling agent is water with a finite, usually rather small yield stress. In the hydrogels of the present invention a network formed through physical interaction between molecules resulting in a thermo-reversible network as the regions of local order are thermally reversible. With respect to rheological parameters this means that the shear storage modulus (G′) needs to be higher than the loss modulus (G″). The G′ is preferentially 10 times higher than G″. Rheological measurements are performed on thermally stable hydrogels.

Optionally, an oil can be added during the formulation of the GA hydrogel. The formulation becomes as structural emulsion.

Depending on the amount of oil is used in the hydrogel formulation, the formulation becomes a oil in water emulsion (volume of water is more than 50 vol % of the formulation) or water in oil emulsion (volume of water is less than 50 vol % of the formulation).

In embodiments of the invention such emulsions are water in oil emulsion with volume of water being more than 50 vol %, more than 25% vol., more than 10 vol. of the formulation. In specific embodiments between 2 and 10 or between 4 and 6 ml oil or added to a 100 ml hydrogel preparation.

Structural emulsion as described hereabove are also referred to as “organogels”. pH determination: The pH within the gel phase of a hydrogel in gel phase cannot be determined, and is therefore measured in the sol phase. Herein the gel itself becomes a liquid (G′~G″) liquid by heating the gel above the degelation temperature and pH is measured at this temperature.

### Hydrogel

In a first aspect, the present invention relates to a glycyrrhizic acid hydrogel with a pH of between 2.5 and 5.0 and comprising an imidazole. The inventors have found that glycyrrhizic acid hydrogels in the specified pH range are more effective against bacterial infections. In particular, glycyrrhizic acid hydrogels at the specified pH range, unlike glycyrrhizic acid hydrogels at a different pH and unlike solutions containing glycyrrhizic acid, have shown to be effective against a broad spectrum of bacterial infections. In comparison, previous glycyrrhizic acid hydrogels and solutions containing glycyrrhizic acid were known to be effective against certain specific bacteria, while ineffective against many others.

Inclusion of an imidazole further enhances the antibacterial and also provides an antifungal activity.

Hydrogels consist of a colloidal or a polymeric network which is expanded in volume by water. Hydrogels can absorb and retain significant amounts of water without dissolving in water. The junction points in the hydrogel network can either be formed by physical interactions or by chemical bonds. In addition, flexible hydrogels are more and more used for curing purposes, especially in wound healing context. Glycyrrhizic acid (glycyrrhizin) is known as a gelling agent with anti-inflammatory properties. Moreover, glycyrrhizic acid can also increase the solubility of active compounds such as antibiotics, antifungals, etc. glycyrrhizic acid is a natural compound derived from the roots of liquorice and is considered as safe by the FDA (GRAS).

Organogels are based on a liquid organic phase, resulting in a cross-linked three-dimensional network.

In addition, the applicant found that the pH had a significant impact on the rheological properties of the hydrogel. Without being bound by theory, it is believed these rheological differences can be attributed to the extent to which glycyrrhizic acid is protonated. Glycyrrhizin contains three carboxyl groups, which can be protonated or deprotonated depending on the pH and this has an influence on e.g. the gel strength and the gelation temperature.

At a pH below 2.5, the glycyrrhizic acid hydrogel becomes brittle even at 37° C., resulting in a gel that breaks or cracks easily. Furthermore, at lower pH glycyrrhizic acid and water must be mixed above the gelation range to obtain a homogeneous gel. As the gelation range increases in temperature with decreasing pH, this results in higher heating and energy requirements. These reasons made working at a pH below 2.5 increasingly impractical; as the gel became difficult to produce and apply. As the pH increases, the gelation and degelation temperature decrease, gel strength and brittleness decrease. The sensitivity to changes and/or variations of the rheological properties to time, temperature, pH and glycyrrhizic acid concentration during gelation/degelation increase significantly.

At a pH above 5.0 gelation at room temperature required increasingly long periods of time. While gelation could be sped up by active cooling, this required more energy and equipment. Furthermore, minor deviations in temperature, pH or glycyrrhizic acid concentration had a large impact on the gel properties, including gelation times and gel strength. As a result, working at a pH above 5.0 quickly became increasingly impractical, as the gel became difficult to use and apply.

In the claimed ranges, water and glycyrrhizic acid can be mixed in the sol phase, that is to say above the degelation point, at relatively low temperatures. This is advantageous from an energetic and safety point of view. Upon cooling said sol phase, a stable and soft hydrogel is rapidly formed even at room temperature. This allows quick, safe and relatively easy production and use of the hydrogel. Present application achieves these features through modification of the pH rather than addition of additional gelators or cross-linking agents. This is particularly advantageous as it reduces issues with compatibility and in-vivo degradability of the glycyrrhizic acid hydrogel.

The term “glycyrrhizic acid hydrogel” as used herein refers to a hydrogel of glycyrrhizic acid, its derivatives, and combinations thereof. Synonyms of “glycyrrhizic acid” are “glycyrrhizin” or “glycyrrhizinic acid”.

In a preferred embodiment, the glycyrrhizic acid hydrogel with a pH of between 2.5 and 5.0 comprises 2.5 to 25 wt. % of a molecule with formula (I) as depicted below or a pharmaceutically acceptable salt or solvate thereof:

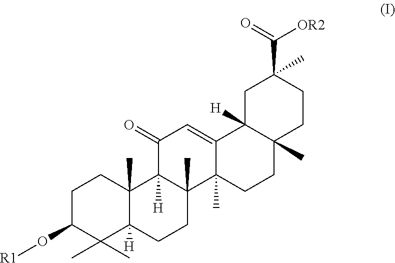

wherein R2 is H or a C1 to C20 alkyl chain;
and R1 is H or a glucuronic acid moiety depicted in formula (II)

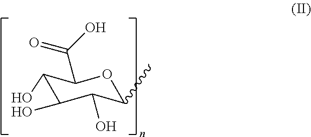

wherein n=1 to 10, [symbol]
wherein the symbol in formula (II) depicts the binding between the glucuronic acid comprising moiety and (I),
wherein if n=1, the glucuronic acid moiety is a glucuronic acid monomer, wherein if n>1, the glucuronic acid comprising moiety is an alfa-1,2-linked glucuronic acid oligomer. In a preferred embodiment, R2 is a glucuronic acid moiety and n is 2.

In a preferred embodiment, R2 is H.

In the most preferred embodiment, R1 and R2 are H as depicted in the below formula (III)

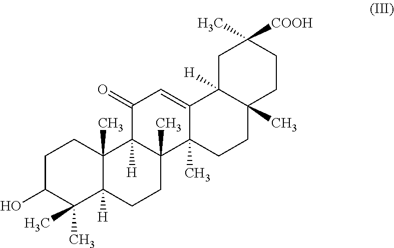

In a preferred embodiment, the invention provides a glycyrrhizic acid hydrogel comprising 2.5 to 25 wt. % of glycyrrhizic acid, a pharmaceutically acceptable salt or solvate thereof, said hydrogel having a pH of between 2.5 and 5.0. In a further preferred embodiment, the glycyrrhizic acid hydrogel comprises 2.5 to 25 wt. % of a pharmaceutically acceptable salt of glycyrrhizic acid. Suitable pharmaceutically acceptable salts thereof may include alkali metal salts, e.g. sodium or potassium salts; alkaline earth metal salts, e.g. calcium or magnesium salts; and salts formed with suitable organic ligands, e.g. ammonium salts. Most preferably, the glycyrrhizic acid hydrogel comprises 2.5 to 25 wt. % of glycyrrhizic acid monoammonium salt.

In a preferred embodiment, the concentration of glycyrrhizic acid, a pharmaceutically acceptable salt or solvate thereof in said glycyrrhizic acid hydrogel is between 2 wt. % to 15 wt. %, more preferably between 2.5 and 15 wt. %, more preferably between 2.5 and 12 wt. %, more preferably between 2.5 and 10 wt. %, more preferably between 2.5 and 9 wt. %, more preferably between 3 and 8 wt. %, more preferably between 3 and 7 wt. %, more preferably between 4 and 7 wt. %, more preferably between 4 and 6 wt. %, most preferably about 5 wt. %. Advantageously, these concentrations of glycyrrhizic acid, a pharmaceutically acceptable salt or solvate thereof in water, at the pH range of 2 to 5 and in the absence of additional gelators results in a hydrogel with a gelation point between 4° and 60° C. Advantageously compared to other hydrogels there is no need for an additional gelator, as glycyrrhizic acid acts as the gelator, thereby reducing toxicity and avoiding an immune response. Furthermore, it allows the hydrogel to be fully degradable and promotes independent API formulation by the physician.

The pH of the hydrogel can be modified to fall within the desired range using any suitable means. In a preferred embodiment, the pH is modified through the addition of pharmaceutically acceptable pH modifiers. In a preferred embodiment, the pH may be buffered in the desired range. This can be advantageous to ensure the pH remains within the desired range when the hydrogel is modified and/or applied. Preferably, pharmaceutically acceptable pH modifiers are chosen from pharmaceutically acceptable acids, pharmaceutically acceptable salts, pharmaceutically acceptable bases and pharmaceutically acceptable buffers. Pharmaceutically acceptable acids include hydrochloric acid, hydrobromic acid, sulphuric acid, phosphoric acid, nitric acid, acetic acid, maleic acid, fumaric acid, lactic acid, tartaric acid, citric acid and gluconic acid. Pharmaceutically acceptable salts include: hydrochloride, hydrobromide, sulphate, phosphate, nitrate, acetate, maleate, fumarate, lactate, sodium hydroxide, potassium hydroxide, magnesium hydroxide, calcium hydroxide and aqueous ammonia NH₄OH. It is noted that these pH modifiers are used in a hydrogel and in order to obtain and/or maintain the pH in the desired range. In other words, these pH modifiers are heavily diluted and used in an aqueous medium. Pharmaceutically acceptable bases are suitable to adjust the pH as well as buffering the hydrogel at a desired pH; but it is noted that the desired pH remains well within the acidic range (i.e. lower than 5). The most preferred pH modifiers are chosen from the list of: HCl, HBr, NaOH and NH₄OH.

In a further preferred embodiment, the present invention provides a glycyrrhizic acid hydrogel essentially consisting of, preferably consisting of:
2.5 to 25 wt. % of glycyrrhizic acid, a pharmaceutically acceptable salt or solvate thereof;
pharmaceutically acceptable pH modifiers; in an amount so said hydrogel has a pH of between 2.5 and 5.0;
an imidazole, and
water.

In another preferred embodiment, the present invention provides a glycyrrhizic acid hydrogel essentially consisting of, preferably consisting of:
2.5 to 25 wt. % of glycyrrhizic acid, a pharmaceutically acceptable salt or solvate thereof;
pharmaceutically acceptable pH modifiers chosen from: hydrochloric acid, hydrobromic acid, sulphuric acid, phosphoric acid, nitric, acetic acid, maleic acid, fumic acid, lactic acid, tartaric acid, citric acid, gluconic acid, hydrochloride salts, hydrobromide salts, sulphate salts, phosphate salts, nitrate salts, acetate salts, maleate salts, fumarate salts, lactate salts, tartrate salts, citrate salts gluconate salts, sodium hydroxide, potassium hydroxide, magnesium hydroxide, calcium hydroxide and aqueous ammonia NH₄OH; in an amount so said hydrogel has a pH of between 2.5 and 5.0;
an imidazole, and
water.

In a preferred embodiment, the concentration of glycyrrhizic acid, a pharmaceutically acceptable salt or solvate thereof in said glycyrrhizic acid hydrogel is between 2 wt. % to 25 wt. %. More preferably, the concentration of glycyrrhizic acid, a pharmaceutically acceptable salt or solvate thereof is between 2.5 and 25 wt. %, more preferably the hydrogel comprises of 2.5%-3%; 2.5%-3.5%; 2.5%-4%; 2.5%-4.5%; 2.5%-5%; 2.5%-5.5%; 2.5%-6%; 2.5%-6.5%; 2.5%-7%; 2.5%-7.5%; 2.5%-8%; 2.5%-8.5%; 2.5%-9%; 2.5%-9.5%; 2.5%-10%; 2.5%-10.5%; 2.5%-11%; 2.5%-11.5%; 2.5%-12%; 2.5%-12.5%; 2.5%-13%; 2.5%-13.5%; 2.5%-14%; 2.5%-14.5%; 2.5%-15%; 2.5%-16%; 2.5%-17%; 2.5%-18%; 2.5%-19%; 2.5%-20%; 2.5%-21%; 2.5%-22%; 2.5%-23%; 2.5%-24%; 2.5%-25%; 3%-3.5%; 3%-4%; 3%-4.5%; 3%-5%; 3%-5.5%; 3%-6%; 3%-6.5%; 3%-7%; 3%-7.5%; 3%-8%; 3%-8.5%; 3%-9%; 3%-9.5%; 3%-10%; 3%-10.5%; 3%-11%; 3%-11.5%; 3%-12%; 3%-12.5%; 3%-13%; 3%-13.5%; 3%-14%; 3%-14.5%; 3%-15%; 3%-16%; 3%-17%; 3%-18%; 3%-19%; 3%-20%; 3%-21%; 3%-22%; 3%-23%; 3%-24%; 3%-25%; 3.5%-4%; 3.5%-4.5%; 3.5%-5%; 3.5%-5.5%; 3.5%-6%; 3.5%-6.5%; 3.5%-7%; 3.5%-7.5%; 3.5%-8%; 3.5%-8.5%; 3.5%-9%; 3.5%-9.5%; 3.5%-10%; 3.5%-10.5%; 3.5%-11%; 3.5%-11.5%; 3.5%-12%; 3.5%-12.5%; 3.5%-13%; 3.5%-13.5%; 3.5%-14%; 3.5%-14.5%; 3.5%-15%; 3.5%-16%; 3.5%-17%; 3.5%-18%; 3.5%-19%; 3.5%-20%; 3.5%-21%; 3.5%-22%; 3.5%-23%; 3.5%-24%; 3.5%-25%; 4%-4.5%; 4%-5%; 4%-5.5%; 4%-6%; 4%-6.5%; 4%-7%; 4%-7.5%; 4%-8%; 4%-8.5%; 4%-9%; 4%-9.5%; 4%-10%; 4%-10.5%; 4%-11%; 4%-11.5%; 4%-12%; 4%-12.5%; 4%-13%; 4%-13.5%; 4%-14%; 4%-14.5%; 4%-15%; 4%-16%; 4%-17%; 4%-18%; 4%-19%; 4%-20%; 4%-21%; 4%-22%; 4%-23%; 4%-24%; 4%-25%; 4.5%-5%; 4.5%-5.5%; 4.5%-6%; 4.5%-6.5%; 4.5%-7%; 4.5%-7.5%; 4.5%-8%; 4.5%-8.5%; 4.5%-9%; 4.5%-9.5%; 4.5%-10%; 4.5%-10.5%; 4.5%-11%; 4.5%-11.5%; 4.5%-12%; 4.5%-12.5%; 4.5%-13%; 4.5%-13.5%; 4.5%-14%; 4.5%-14.5%; 4.5%-15%; 4.5%-16%; 4.5%-17%; 4.5%-18%; 4.5%-19%; 4.5%-20%; 4.5%-21%; 4.5%-22%; 4.5%-23%; 4.5%-24%; 4.5%-25%; 5%-5.5%; 5%-6%; 5%-6.5%; 5%-7%; 5%-7.5%; 5%-8%; 5%-8.5%; 5%-9%; 5%-9.5%; 5%-10%; 5%-10.5%; 5%-11%; 5%-11.5%; 5%-12%; 5%-12.5%; 5%-13%; 5%-13.5%; 5%-14%; 5%-14.5%; 5%-15%; 5%-16%; 5%-17%; 5%-18%; 5%-19%; 5%-20%; 5%-21%; 5%-22%; 5%-23%; 5%-24%; 5%-25%; 6%-7%; 6%-8%; 6%-9%; 6%-10%; 6%-11%; 6%-12%; 6%-13%; 6%-14%; 6%-15%; 6%-16%; 6%-17%; 6%-18%; 6%-19%; 6%-20%; 6%-21%; 6%-22%; 6%-23%; 6%-24%; 6%-25%; 7%-8%; 7%-9%; 7%-10%; 7%-11%; 7%-12%; 7%-13%; 7%-14%; 7%-15%; 7%-16%; 7%-17%; 7%-18%; 7%-19%; 7%-20%; 7%-21%; 7%-22%; 7%-23%; 7%-24%; 7%-25%; 8%-9%; 8%-10%; 8%-11%; 8%-12%; 8%-13%; 8%-14%; 8%-15%; 8%-16%; 8%-17%; 8%-18%; 8%-19%; 8%-20%; 8%-21%; 8%-22%; 8%-23%; 8%-24%; 8%-25%; 9%-10%; 9%-11%; 9%-12%; 9%-13%; 9%-14%; 9%-15%; 9%-16%; 9%-17%; 9%-18%; 9%-19%; 9%-20%; 9%-21%; 9%-22%; 9%-23%; 9%-24%; 9%-25%; 10%-12%; 10%-14%; 10%-16%; 10%-18%; 10%-20%; 10%-22%; 10%-25%; 12%-15%; 12%-18%; 12%-21%; 12%-25%; 14%-18%; 14%-21%; 14%-23%; 14%-25%; 16%-19%; 16%-21%; 16%-23%; 16%-25%; or 25% glycyrrhizic acid, a pharmaceutically acceptable salt or solvate thereof in water. The wording “% of a molecule” as described herein refers to wt. % or percentage by weight.

In a preferred embodiment, the glycyrrhizic acid hydrogel has a temperature lower than 40° C. That is to say, the glycyrrhizic acid hydrogel is a stable hydrogel at temperatures lower than 40° C. In a preferred embodiment, the glycyrrhizic acid hydrogel has a gelation point higher than 40° C. In a preferred embodiment, the glycyrrhizic acid hydrogel has a degelation point higher than 40° C. It is strongly preferred that the degelation point of the hydrogel is higher than the body temperature of the mammal to be treated for a wound otherwise the hydrogel will become liquid when applied onto the wound. Present application shows that the hydrogel form has significant beneficial effects for use in treatment or prevention of bacterial or fungal infections than a liquid form. For example the gelation point for the 2.5% Glycyrrhizic acid hydrogel was determined to be 40° C. in the experimental setup of the examples of the present invention, while the gelation point for the 5% glycyrrhizic acid hydrogel was determined to be at 45° C., under these experimental conditions.

The “gelation point” or “gelation temperature” as used herein is defined as the temperature at which the elastic modulus G′ becomes larger than the loss modulus G″, measured while cooling down at a rate of 0.01° C./s. The “degelation point” or “degelation temperature” is defined as the temperature at which the loss modulus G″ becomes larger than the elastic modulus G′, measured while heating up at a rate of 0.01° C./s.

Glycyrrhizic acid hydrogels gelation and degelation show hysteresis, so the gelation and degelation point and degelation point are not a single value. The “gelation range” or “degelation range” as used herein is the interval defined by the gelation point and the degelation point; both measured with a temperature sweep of 0.01° C./s.

Preferably, the gelation and degelation range lies entirely above the body temperature of the mammal to be treated, more preferably above 40° C. In a further preferred embodiment, the degelation range lies entirely between 4° and 70° C., more preferably the degelation range lies entirely between 4° and 60° C., more preferably the degelation range lies entirely between 4° and 50° C. The minimum temperature of 40° C. is beneficial to ensure the hydrogel remains a hydrogel, which is highly desirable for its antibacterial and wound-dressing functions. The maximum temperature is beneficial to allow easy, safe and less energy-intensive processability of the hydrogel in its sol phase.

“Imidazole” in the context of the present invention may be any type of imidazole known in the art or salts thereof, including nitroimidazoles. Non-limiting examples of imidazoles include miconazole, clotrimazole, ketoconazole and metronidazole.

In a further preferred embodiment, the hydrogel may further comprise additional ingredients. Particularly beneficial ingredients include antiseptics, antibiotics, antimycotics, antivirals, parasiticide as well as active pharmaceutical ingredients in particular those suitable for topical treatments and dermatological conditions.

In a preferred embodiment, the hydrogel comprises an antiseptic chosen from the list of: benzalkonium chloride, cetrimide, chlorhexidine, chloroxylenol, clorophene, dequalinium chloride, domiphen bromide, ethanol, hexamidine, sodium hypochlorite, nitrofural, povidone iodine, tosylchloramide, hydrogen peroxide and combinations thereof. Most preferably, the antiseptic is dequalinium chloride or domiphen bromide.

In a preferred embodiment, the hydrogel comprises an antibiotic chosen from the list of: fusidic acid, mupirocin, terramycin, neobacitracin, polymyxin, sulfadiazine and combinations thereof, more preferably the antibiotic is fusidic acid.

In a preferred embodiment, the hydrogel comprises a further antimycotic chosen from the list of: amorolophine, cyclopirox, seleniumsulfide, terbinafine and combinations thereof.

In a preferred embodiment, the hydrogel comprises an antiviral agent chosen from the list of: aciclovir, docosanol, penciclovir and combinations thereof.

In a preferred embodiment, the hydrogel comprises an antiparasitic, more preferably said antiparasitic is permethrin.

In a preferred embodiment, the hydrogel comprises a corticosteroid chosen from the list of: clobetasol, betamethasone, methylprednisolone, hydrocortisone, mometasone, diflucortolone and combinations thereof.

In a preferred embodiment, the hydrogel comprises an anti-histamine. Preferably the hydrogel comprises diphenhydramine.

In a preferred embodiment, the hydrogel comprises an API suitable to treat acne chosen from the list of: benzoyl peroxide, clindamycin, erythromycin, azelaic acid, adapalene, trifarotene and combinations thereof.

In a preferred embodiment, the hydrogel comprises an API suitable to treat rosaceae chosen from the list of: brimonidine, ivermectin, metronidazole and combinations thereof, most preferably metronidazole.

In a preferred embodiment, the hydrogel comprises an API suitable to treat psoriasis chosen from the list of: tacalcitol, salicylic acid, calcipotriol and combinations thereof, most preferably salicylic acid.

In a preferred embodiment the hydrogel comprises enzymes in particular collagenase.

In a preferred embodiment, the hydrogel comprises protectives chosen from the list of: ichthammol, boric acid and combinations thereof.

In a preferred embodiment, the hydrogel comprises immunomodulators chosen from the list of: imiquimod, pimecrolimus, tacrolimusand combinations thereof.

In a preferred embodiment, the hydrogel comprises an anaesthetic, preferably lidocaine.

In a preferred embodiment, the hydrogel further comprises: fluorouracil, 5-aminolevulinic acid, methylaminolevulinate, tirbanibulin or a combination thereof.

### Treatment

In a second aspect, the present invention provides a glycyrrhizic acid hydrogel comprising an imidazole for use in the treatment or prevention bacterial and fungal infections, preferably skin and soft tissue infections. In a preferred embodiment, the present invention provides a glycyrrhizic acid hydrogel comprising an imidazole with a pH below 5.0, for use in the treatment or prevention of a bacterial or fungal infection. In a more preferred embodiment, the invention provides a glycyrrhizic acid hydrogel comprising 2.5 to 25 wt. % of glycyrrhizic acid, a pharmaceutically acceptable salt or solvate thereof, and an imidazole, said hydrogel having a pH of between 2.5 and 5.0, for use in the treatment or prevention of a bacterial or fungal infection.

In a further aspect, the present invention provides a method of treating or preventing a bacterial or fungal infection in an individual comprising the step of administering an effective amount of the glycyrrhizic acid hydrogel comprising an imidazole. In a preferred embodiment, the present invention provides a method of treating or preventing a bacterial or fungal infection in an individual comprising the step of administering an effective amount of the glycyrrhizic acid hydrogel, a pharmaceutically acceptable salt or solvate thereof, and comprising an imidazole with a pH of between 2.5 and 5.0. The applicant has surprisingly found that acidifying a glycyrrhizic acid hydrogel increases the activity spectrum of said gel against bacterial infection, as well as modifying the rheological properties of the gel.

In a preferred embodiment, the glycyrrhizic acid hydrogel has a pH lower than 5.0, more preferably a pH lower than 4.9, more preferably a pH lower than 4.8, more preferably a pH lower than 4.85, more preferably a pH lower than 4.8, more preferably a pH lower than 4.75, more preferably a pH lower than 4.7, more preferably a pH lower than 4.6, more preferably a pH lower than 4.5, more preferably a pH lower than 4.4, more preferably a pH lower than 4.3, more preferably a pH lower than 4.2. Preferably, the glycyrrhizic acid hydrogel has a pH of at least 1,0, more preferably a pH of at least 2.0, more preferably a pH of at least 2.1, more preferably a pH of at least 2.2, more preferably a pH of at least 2.3, more preferably a pH of at least 2.4, more preferably a pH of at least 2.5, more preferably a pH of at least 2.6, more preferably a pH of at least 2.7, more preferably a pH of at least 2.8, more preferably a pH of at least 2.9, more preferably a pH of at least 3.0, more preferably a pH of at least 3.1, more preferably a pH of at least 3.2.

In a preferred embodiment, the glycyrrhizic acid hydrogel has a pH between 1.0 and 5.0, more preferably a pH between 2.0 and 5.0, more preferably a pH between 2.5 and 5.0, more preferably a pH between 2.6 and 4.9, more preferably a pH between 2.7 and 4.8, more preferably a pH between 2.8 and 4.8.

In a further preferred embodiment, the glycyrrhizic acid hydrogel has a pH between 4.0 and 4.4, more preferably a pH between 4.1 and 4.3, most preferably a pH of about 4.2. In a further preference, this hydrogel is for use in the treatment or prevention of a bacterial invention, wherein the bacterial infection is of MRSA, Staphylococcus epidermidis, Acinetobacter baumanni, E. faecalis or S. pyogenes. More preferably, the bacterial infection is of MRSA, Acinetobacter baumannii and Streptococcus pyogenes. This particular pH range was found to be the most effective against a bacterial infection of MRSA, Acinetobacter baumannii and Streptococcus pyogenes. Furthermore, this pH range was found very effective against bacterial infection of MRSA, Acinetobacter baumannii and Streptococcus pyogenes.

In a further preferred embodiment, the glycyrrhizic acid hydrogel has a pH between 3.6 and 4.0, more preferably a pH between 3.7 and 3.7, most preferably a pH of about 3.8. In a further preference, this hydrogel is for use in the treatment or prevention of a bacterial invention, wherein the bacterial infection is of Pseudomonas aeruginosa. This particular pH range was found to be the most effective against a bacterial infection of Pseudomonas aeruginosa.

In another preferred embodiment, the glycyrrhizic acid hydrogel has a pH between 2.8 and 3.2, more preferably a pH between 2.9 and 3.2, most preferably a pH of about 3. In a further preference, this hydrogel is for use in the treatment or prevention of a bacterial infection, wherein the bacterial infection is of Pseudomonas aeruginosa, Escherichia coli, Klebsiella pneumoniae, Klebsiella aerogenes or Enterococcus faecalis. Most preferably, this hydrogel is for use in the treatment or prevention of a bacterial infection wherein the bacterial infection is of Escherichia coli, Klebsiella pneumoniae or Klebsiella aerogenes. This particular pH range was found to be the most effective against a bacterial infection of found to be the most effective against a bacterial infection of Escherichia coli, Klebsiella pneumoniae or Klebsiella aerogenes. Furthermore, this pH range was found very effective against bacterial infection of Pseudomonas aeruginosa, Escherichia coli, Klebsiella pneumoniae, Klebsiella aerogenes or Enterococcus faecalis.

In a preferred embodiment, the hydrogel with imidazole is for topical use. In a further preferred embodiment, the hydrogel is for use in the treatment or prevention of a bacterial or fungal infection, wherein the hydrogel is for topical use.

In a further preferred embodiment, the hydrogel with imidazole is for topical use on chronic wounds. The term “chronic wound” refers to a wound that has not healed. Wounds that do not heal within 6 weeks, for example, are considered chronic. Chronic wounds include, for example, pressure ulcers, decubitus ulcers, diabetic ulcers including diabetic foot and leg ulcers, slow or non-healing venous ulcers, venous stasis ulcers, arterial ulcers, vasculitic ulcers, burn ulcers, trauma-induced ulcers, infectious ulcers, mixed ulcers, and pyoderma gangrenosum. The chronic wound may be an arterial ulcer that comprises ulcerations resulting from complete or partial arterial blockage. The chronic wound may be a venous or venous stasis ulcer that comprises ulcerations resulting from a malfunction of the venous valve and the associated vascular disease. Bacterial and fungal infections commonly occur in chronic wounds as they provide suitable entry and growth locations for bacteria. The glycyrrhizic acid hydrogel of present invention advantageously may be used to treat or prevent bacterial infections over a broad spectrum of bacteria.

In another preferred embodiment, the hydrogel with imidazole is for internal use. In a further preferred embodiment, the hydrogel is for use in the treatment or prevention of a bacterial or fungal infection, wherein the hydrogel is for internal use.

In another preferred embodiment, the hydrogel with imidazole is applied as a coating on a medical device. In a further preferred embodiment, the hydrogel with imidazole is for use in the treatment or prevention of a bacterial or fungal infection wherein the hydrogel is applied as a coating on a medical device.

A non-limited list of conditions and bacteria to be treated with the hydrogels of the present invention are listed in table 1.

TABLE 1
Examples of etiological risk factors for skin and soft
tissue infections and their associated bacterial causes

Risk factor Associated etiological
pathogen

| | |
|---|---|
| Diabetes mellitus | Staphylococci, group B streptococci, |
| | Acinetobacter baumannii, Eschericia coli |
| Cirrhosis | Klebsiella pneumoniae, Klebsiella |
| | aerogenes, |
| Escherichia coli, Acinetobacter | Pseudomonas aeruginosa |
| baumannii Neutropenia | |
| Hot tub exposure/ loofah sponge | Pseudomonas aeruginosa |
| IV drug abuse | Methicillin-resistant Staphylococcus |
| | aureus (MRSA), Pseudomonas |
| | aeruginosa |

### EXAMPLES

### Example 1: Decreasing the pH of the Glycyrrhizic Acid Hydrogel Increases the Activity Spectrum

The circles in FIG. 1 represent the number of pathogens (9 tested in total) of which the growth is completely inhibited by the specific hydrogel (defined according to glycyrrhizic acid concentration and gel surface pH). The circle size is proportional to the number of pathogens of which the growth is completely inhibited. The glycyrrhizic acid concentration is calculated based on the percentage of glycyrrhizic acid in the gel and the amount of this gel used in the assay. Note that the pH at the gel surface is lower than the initial pH of the growth medium on top of the gel due to the acidifying effect of the hydrogel. This pH difference is dependent of the pH of the hydrogel, the percentage of glycyrrhizic acid in the hydrogel and the amount of hydrogel used. The different hydrogels are indicated by color: white circles represent the hydrogel A, grey circles represent hydrogel B and black circles represent the hydrogel C. The 2 grey dots at pH 7.0 in the plot represent the (lack of) activity of glycyrrhizic acid in solution. Of all nine pathogens tested, only Streptococcus pyogenes was inhibited by 50 mg/mL glycyrrhizic acid in solution.

The black triangle at pH 6.5 in the plot represent the activity of the hydrogel prepared as described in (Zhao et al. (2020) cited above). Herein the hydrogel of Zhao et al was prepared according to the instructions of the authors and pH measurement was done as detailed in the methods and materials of the present invention.

### Example 2: Decreasing the pH of the Glycyrrhizic Acid Hydrogel Increases the Antibacterial Activity

The below table summarises the antibacterial effect (no effect, bacteriostatic, bactericidal) of the glycyrrhizic acid hydrogels on different pathogens tested.

TABLE 2
Overview of the antibacterial effect (no effect, bacteriostatic, bactericidal)
of the glycyrrhizic acid hydrogels on different pathogens tested.

| | GLY in solution | Hydrogel A | Hydrogel B | Hydrogel C |
|---|---|---|---|---|
| MRSA | No effect | Bactericidal | Bactericidal | Bactericidal |
| S. epidermidis | No effect | Bacteriostatic | Bactericidal | Bactericidal |
| A. baumannii | No effect | Bactericidal | Bactericidal | Bactericidal |
| P. aeruginosa | No effect | No effect | Bacteriostatic | Bactericidal |
| E. coli | No effect | No effect | No effect | Bactericidal |
| | | | | (>50 mg/mL) |
| K. | No effect | No effect | No effect | Bactericidal |
| pneumoniae | | | | (>50 mg/mL) |
| K. aerogenes | No effect | No effect | No effect | Bactericidal |
| | | | | (>50 mg/mL) |
| Streptococcus | Bacteriostatic | Bactericidal | Bactericidal | Bactericidal |
| Enterococcus | No effect | Bacteriostatic | Bacteriostatic | Bactericidal |

### Example 3: Growth Inhibiting Activity of Hydrogel A

Hydrogel A shows increased growth inhibiting activity against MRSA, Staphylococcus epidermidis, Acinetobacter baumanni, E. faecalis and S. pyogenes, as compared to the lack of/limited growth inhibiting activity of glycyrrhizic acid in solution or at the corresponding pH associated with the acidification of the environment caused by glycyrrhizic acid hydrogels.

The growth inhibiting effect of 25 mg/mL glycyrrhizic acid in solution and 0.5 mL/mL of 5 wt % hydrogel A, corresponding with a total of 25 mg/ml of glycyrrhizic acid in the system are shown in grey bars with circles. Both are compared to suitable controls (shown in white bars with squares) i.e. growth media with an adjusted pH that is associated with the acidifying effect of glycyrrhizic acid in solution (pH 7,4) and hydrogel A (pH 5,2) but without the presence of glycyrrhizic acid in solution or hydrogel A, respectively. ns: p>0.05, * p<0.05, ** p<0.01, **** p<0.0001.

### Example 4: Bactericidal Activity of Hydrogel A

Hydrogel A shows bactericidal activity against MRSA, Acinetobacter baumannii and Streptococcus pyogenes.

Bactericidal activity is defined as a 3 LOG reduction compared to the inoculum (black bars). The bactericidal effect of 0.5 mL/mL of 5 wt % hydrogel A (shown in grey bars with circles) is compared to growth medium with an adjusted pH (pH 5,2) that is associated with the acidifying effect of hydrogel A (shown in white bars with squares). ns: p>0.05, **** p<0.0001.

### Example 5: Growth Inhibiting Activity of Hydrogel B

Hydrogel B shows increased growth inhibiting activity against Pseudomonas aeruginosa, as compared to the lack of/limited growth inhibiting activity of glycyrrhizic acid in solution or at the corresponding pH associated with the acidification of the environment caused by glycyrrhizic acid hydrogels.

The growth inhibiting effect of 25 mg/mL glycyrrhizic acid in solution and 0.5 mL/mL of 5 wt % hydrogel B, corresponding with a total of 25 mg/mL of glycyrrhizic acid in the system are shown in grey bars with circles. Both are compared to suitable controls (shown in white bars with squares) i.e. growth media with an adjusted pH that is associated with the acidifying effect of glycyrrhizic acid in solution (pH 7,4) and hydrogel B (pH 5) but without the presence of glycyrrhizic acid in solution or hydrogel B, respectively. ns: p >0.05, **** p<0.0001.

### Example 6: Bactericidal Activity of Hydrogel B Shows

Hydrogel B shows bactericidal activity against Staphylococcus epidermidis. Bactericidal activity is defined as a 3 LOG reduction compared to the inoculum (black bars). The bactericidal effect of 0.5 mL/mL of 5 wt % hydrogel B (shown in grey bars with circles) is compared to growth medium with an adjusted pH (pH 5) that is associated with the acidifying effect of hydrogel B (shown in white bars with squares). **** p<0.0001.

### Example 7: Growth Inhibiting Activity of Hydrogel C

Hydrogel C shows increased growth inhibiting activity against Escherichia coli, Klebsiella pneumoniae and Klebsiella aerogenes, as compared to the lack of/limited growth inhibiting activity of glycyrrhizic acid in solution or at the corresponding pH associated with the acidification of the environment caused by glycyrrhizic acid hydrogels.

The growth inhibiting effect of 50 mg/ml glycyrrhizic acid in solution and 0.5 mL/mL of 10 wt % hydrogel C, corresponding with a total of 50 mg/ml of glycyrrhizic acid in the system are shown in grey bars with circles. Both are compared to suitable controls (shown in white bars with squares) i.e. growth media with an adjusted pH that is associated with the acidifying effect of glycyrrhizic acid in solution (pH 7,4) and hydrogel C (pH 4.6) but without the presence of glycyrrhizic acid in solution or hydrogel C, respectively. ns: p >0.05, **** p<0.0001.

### Example 8: Bactericidal Activity of Hydrogel C

Hydrogel C shows bactericidal activity against Pseudomonas aeruginosa, Escherichia coli, Klebsiella pneumoniae, Klebsiella aerogenes and Enterococcus faecalis. Bactericidal activity is defined as a 3 LOG reduction compared to the inoculum (black bars). The bactericidal effect of 0.5 mL/mL of 10 wt % hydrogel C (shown in greybars with circles) is compared to growth medium with an adjusted pH (pH 4.6) that is associated with the acidifying effect of hydrogel C (shown in white bars with squares). **** p<0.0001.

### Examples 9: Materials and Methods

### Strains and Chemicals

Escherichia coli UTI89, Pseudomonas aeruginosa LMG9009, Staphylococcus aureus ATCC6538, Klebsiella pneumoniae ATCC13883, Klebsiella aerogenes ATCC13048, Acinetobacter baumannii RD5SR3. Enterococcus faecalis LMG8148, Streptococcus pyogenes LMG 14237 and clinical isolates of a Staphylococcus epidermidis and a methicillin resistant S. aureus (MRSA), isolated from osteomyelitis patients were used. Prior to the experiments, all strains, except S. pyogenes were inoculated in Lysogeny broth (LB) and incubated overnight on 37° C. (200 rpm). Overnight cultures of S. pyogenes were grown micro-aerophilic in Brain-Heart infusion broth (BHI). Thereafter, the bacteria were normalized to roughly 5*10⁵ CFU mL⁻¹. Lysogeny broth (LB; 10 g L⁻¹ NaCl, 10 g L⁻¹ Tryptone, 5 g L⁻¹ Yeast Extract) was used to create overnight cultures (ONC). Mueller Hinton Broth (MHB; 21 g L⁻¹ MHB) was used for the minimal inhibitory concentration (MIC) assays for all strain except S. pyogenes. For this strain, BHI was used. If LB agar, BHI agar or MHB agar was needed, 15 g L⁻¹ of bacteriological agar was added. MHB or BHI batches with varying pH were made by adding HCl or NaOH to acidify or alkalify the medium to the desired pH-value, respectively. Three different formulations of the glycyrrhizic acid hydrogels were made in dH₂O:

(i) Hydrogel C with pH=3, (ii) Hydrogel B with pH=3.8 and (iii) Hydrogel A with pH=4.2. pH was measured at 70° C. and varied using HCl or NaOH to acidify or alkalify the formulation to the desired pH-value. Moreover, different concentration batches (mass by volume) were used for the hydrogels: 2.5%, 5%, 7,5%, 10%, 15% and 20%. Glycyrrhizic acid in solution—not a hydrogel—was prepared at pH 7,4.

### Growth Inhibition Evaluation

To determine the growth inhibiting activity of glycyrrhizic acid in solution, 100 μL of different 1:2 dilutions of a 100 mg/mL stock solution of glycyrrhizic acid in MHB were added to the wells of a 96 well microtiter plate, after which 100 μL of a 5*105 CFU mL⁻¹ bacteria suspension in MHB was added to each well.

To determine the growth inhibiting activity of the glycyrrhizic acid hydrogels, the hydrogel was liquefied by heating to 70° C. When liquid, varying volumes of the liquid hydrogel (80 μL to 160 μL) were pipetted into a 96 well microtiter plate. After the hydrogel solidified, 120 μL of a 5*105 CFU mL⁻¹ bacteria suspension in MHB was added on top of the hydrogel.

To avoid evaporation, the plates were sealed using a membrane (Greiner Bio-one NV). After 24 hours of incubation (37° C., 200 rpm), the OD595 was measured and relative OD595 was calculated by dividing the OD595 by the average OD595 of the growth control (=growth of cells in MHB without glycyrrhizic acid).

### Biocidal Activity Evaluation

To determine the biocidal activity of glycyrrhizic acid in solution and in hydrogel, at the end of the growth inhibition assay (see above), after incubation, 100 μL of the medium on top of the hydrogel was diluted 10 times in Phosphate saline buffer (PBS; 1.24 g L⁻¹ K₂HPO₄. 0.39 g L⁻¹ KH₂PO₄. 8.8 g L⁻¹ NaCl) and plated out on MHB agar plates by spreading 100 μL of the dilution. After incubating overnight at 37° C., colonies were counted and the colony forming units were determined.

### Rheological Evaluation

The rheological properties of glycyrrhizic acid hydrogels over the pH range of 2 to 5 were evaluated during time, strain, frequency and temperature sweeps on a stress controlled rheometer. Additionally, DSC measurements were performed.

It was found that the strength of the hydrogels decreases with increasing pH. At pH 3.00 and lower, microcracks start forming inside the material. This resulted in a hard but increasingly brittle hydrogel. The differences in rheological properties between 20° C. and 37° C. at pH 3 were small. Gelation starts almost instantly, and the moduli reach steady-state in less than 5 minutes.

Furthermore, mixing of glycyrrhizic acid and water quickly forms an impermeable gel; making it impossible for the mixture to gelate completely. To obtain a homogeneous gel, the mixture should be heated to above its degelation temperature and adequately mixed in the sol phase. As the gelation temperature and degelation temperature both increase with decreasing pH; lower pH results in higher heating requirements. These reasons made working with a hydrogel with pH below 2.5 impractical. As the pH increases, the hydrogels become softer and less brittle. Furthermore, it takes longer for the samples to gelate. In other words, it takes longer for the moduli to reach steady-state with increasing pH. The gelation temperature decreases with increasing pH. At a pH above 5.35 and a glycyrrhizic acid concentration of 10 wt. %, the gelation temperature is under room temperature. The gels are relatively frequency independent at every pH.

At pH near 5 and above, the effects of temperature and time on gel formation become increasingly large. For example, at a (monoammonium) glycyrrhizin concentration of 10 wt. % in water, adjusted in pH with HCl and NH₄OH, there is an enormous difference in moduli and gelation time between pH 4.70 and pH 4.85 at 20° C. While both pH result in a soft hydrogel in steady state; reaching G′ of 100 Pa takes 5 minutes 30 seconds at a pH of 4.70. Reaching the same G′ of 100 Pa takes 75 minutes at pH 4.85.

Due to the drastically increasing gelation times as well as the sensitivity of the gel to differences in pH, temperature and glycyrrhizic acid concentration; using glycyrrhizic acid hydrogels at a pH above 5 becomes increasingly impractical. Gelation may be sped up through cooling, but this increases the necessary equipment and energy requirements for preparation. Furthermore, this was found to exacerbates issues with a high temperature sensitivity when the hydrogel is applied to a wound. The rapid substantial decrease in gel strength due to the increased temperature resulted in difficult handling.

### Example 10. Formulation of Imidazoles in Glycyrrhizic Acid-Based Hydro- and Organo-Gels

### Miconazole Nitrate (2 wt. %)+Glycyrrhizic Acid (5 wt. %)

Miconazole nitrate (Merck) was added to deionized water while mixing with a high shear mixer, resulting in 2 wt. % miconazole. The pH is of the solution was adjusted to pH 7 via addition of a base (KOH, NaOH). Subsequently the mixture was heated to 70° C. and glycyrrhizic acid (Acros Organics NV) was added stepwise up to 5 wt. % glycyrrhizic acid, while swerving. The pH was subsequently lowered to pH 4 by addition of HCl. Upon cooling to 4° C. during 12 h, a miconazole-glycyrrhizic acid hydrogel was formed.

### Miconazole Nitrate (2 wt. %)+Glycyrrhizic Acid (5 wt. %)+Oil (5 wt %):

Miconazole nitrate (Merck) was added to deionized water while mixing with a high shear mixer, resulting in 2 wt. % miconazole. The pH of the solution was adjusted to pH 7 via addition of a base (KOH, NaOH). Subsequently the mixture was heated to 70° C. and glycyrrhizic acid (Acros Organics NV) was added stepwise up to 5 wt. % glycyrrhizic acid, while swerving. The pH was subsequently lowered to pH 4 by addition of HCl. Next, oil was added to the solution and mixed by a high shear mixer for 2 min at 13500 rpm during continuous cooling. Upon cooling to 4° C. during 12 h, a miconazole glycyrrhizic acid organogel was formed.

### Ketoconazole (2 wt. %)+Glycyrrhizic Acid (5 wt. %)

Ketoconazole (ketonazole) (TCI Chemicals) was added to deionized water while mixing with a high shear mixer, resulting in 2 wt. % ketonazole. Subsequently the mixture was heated to 70° C. and glycyrrhizic acid (Acros Organics NV) was added stepwise up to 5 wt. % glycyrrhizic acid, while swerving. The pH was subsequently lowered to pH 4 by addition of HCl. Upon cooling to 4° C. during 12 h (overnight), a ketonazole-glycyrrhizic acid hydrogel was formed.

### Clotrimazole (0.5 wt. %)+Glycyrrhizic Acid (5 wt %)

Clotrimazole (Merck) was added to deionized water while mixing with a high shear mixer, resulting in 0.5 wt. % Clotrimazole. Subsequently the mixture was heated to 70° C. The pH of the solution was adjusted to pH 2 via addition of HCl. Subsequently glycyrrhizic acid (Acros Organics NV) was added stepwise up to 5% glycyrrhizic acid, while swerving. The pH was subsequently lowered to pH 4 by addition of HCl. Upon cooling to 4° C. during 12 h (overnight), a Clotrimazole-glycyrrhizic acid hydrogel is formed.

### Example 11. Assessment of Fungicidal and Bactericidal Activity of the Creams and Gels

### Fungicidal Activity

Candida spp. (Candida albicans and Candida glabrata) were grown overnight at 30° C. in YPD (yeast extract (10 g/L; LabM, UK), peptone (20 g/L; LabM, UK) and glucose (20 g/L; Sigma-Aldrich, USA)). Candida cells (OD_{600 nm}=1, 0.1 and 0.01, equaling 10⁷, 10⁶ and 10⁵ cells/mL, respectively) were spotted on Durapore® membrane filters, 0.45 μm (Millipore), resulting in 10⁶, 10⁵ and 10⁴ spotted cells per filter, respectively. Filters were transferred to sterile 6-well in which the wells were coated with different creams/gels (typically 2 ml). The filter is applied so that the side on which the cells were applied is on top. Thus the cells were not in direct contact with the creams/gels. The 6-well plates were incubated during 1 hour at 37° C. after which the filters were transferred to YPD agar plates, with the top side contacting the agar, allowing the cells to be in direct contact with the plates during 1 hour at room temperature. YPD agar plates were incubated overnight at 37 C.°. Cells grown on YPD agar plates were resuspended in 1 ml sterile, distilled water. In a spot assay, 10-fold dilution series of these resuspended yeast cells were prepared in sterile, distilled water, after which 5 μL was spotted on YPD plates and grown overnight at 37° C. Pictures were taken to evaluate the effect of the creams/gels on the cells.

### Bactericidal Activity

Staphylococcus aureus ATCC6538 was grown overnight at 37° C. in Mueller Hinton (MH) broth (Fisher Scientific). Staphylococcus cells (10⁶ and 10⁵ cells/mL) were spotted on Durapore® membrane filters, 0.45 μm (Millipore), resulting in 105 and 10⁴ spotted cells per filter, respectively.

Filters were transferred to sterile 6-well in which the wells were coated with different creams/gels (typically 2 ml). The filter is applied so that the side on which the cells were applied is on top. Thus the cells were not in direct contact with the creams/gels. The 6-well plates were incubated during 1 hour at 37° C. after which the filters were transferred to MH agar plates, with the top side contacting the agar, allowing the cells to be in direct contact with the plates during 1 hour at room temperature.

MH agar plates were incubated overnight at 37 C.°. Cells grown on MH agar plates were resuspended in 1 ml sterile, distilled water. In a spot assay, 10-fold dilution series of these resuspended yeast cells were prepared in sterile, distilled water, after which 5 μL was spotted on MH agar plates and grown overnight at 37° C. Pictures were taken to evaluate the effect of the creams/gels on the cells.

Gardnerella vaginalis strain ATCC 14018 was grown routinely on Columbia Blood Medium (2.3% peptone (International Medical Products NV); 0.1% starch (Merck Millipore), 0.5% sodium chloride (TCI Europe NV) supplemented with 5% defibrinated sheep blood) agar plates at 37° C., anaerobically. Brain Heart Infusion (BHI) broth, purchased from Bio-Rad laboratories was used as overnight culture medium. Gardnerella cells (10⁴ 5×10³ and 10³ cells/mL) were spotted on Durapore® membrane filters, 0.45 μm (Millipore), resulting in 1000, 500 and 10⁰ spotted cells per filter, respectively. Filters were transferred to sterile 6-well in which the wells were coated with different creams/gels. The cells were not in direct contact with the creams/gels. The 6-well plates were incubated during 1 hour at 37° C. after which the filters were transferred to Columbia Blood agar plates, allowing the cells to be in direct contact with the plates during 1 hour at room temperature. Columbia Blood agar plates were incubated overnight at 37 C.°, anaerobically. Cells grown on Columbia Blood agar plates were resuspended in 1 ml sterile, saline solution. In a spot assay, 10-fold dilution series of these resuspended bacterial cells were prepared in sterile, saline solution, after which 5 μL was spotted on Columbia Blood agar plates and grown overnight at 37° C., anaerobically. Pictures were taken to evaluate the effect of the creams/gels on the cells.

### Example 12. Antifungal Activity of Miconazole in Different Gels

Filters with Candida albicans cells were applied on glycyrrhizic acid hydrogels and glycyrrhizic acid organogels, with and without 2 wt. % miconazole nitrate as prepared in Example 1, and tested for antifungal activity according to the methodology described in Example 2.

A commercial preparation of Daktarin™ crème is used as a reference [2 wt. % miconazole nitrate in PEG-6-(PEG-32) glycol stearate, macrogolglycerol oleate, liquid paraffin, butylhydroxyanisole, benzoic acid and water.] The glycyrrhizic acid hydrogel and glycyrrhizic acid-organogel vehicle without miconazole are less effective that the commercial miconazole crème.

Inclusion of miconazole gave an antifungal activity which is significantly better than the commercial miconazole crème.

Herein the hydrogel formulation performs better that the organogel formulation. Indeed, with the highest concentration of applied fungal cell (1 million cells), no cells are seen with the glycyrrhizic acid-hydrogel, while cells are still visible with the organogel.

### Example 13. Antibacterial Activity of Miconazole

Filters with Staphylococcus aureus cells were applied on glycyrrhizic acid hydrogels and glycyrrhizic acid organogels, with and without 2 wt. % miconazole nitrate as prepared above, and tested for antifungal activity according to the methodology described above.

The glycyrrhizic acid hydrogel and glycyrrhizic acid-organogel vehicle without miconazole are less effective that the commercial miconazole crème when 10.000 cells were used. However, using ten times more bacteria (100.000), the difference between the commercial crème and the gel is less clear.

Inclusion of miconazole gave an bacterial activity which is significantly better than the commercial miconazole creme, especially for the hydrogel formulation where even with the highest concentration of S. aureus there are no bacterial visible.

Compared with the organogel formulation, the commercial creme is more active at lower bacterial load (10.000 bacteria).

Herein the hydrogel formulation performs better that the organogel formulation.

Indeed, with the highest concentration of applied fungal cell (1 million cells), no cells are seen with the glycyrrhizic acid-hydrogel, while cells are still visible with the organogel.

Equally, miconazole formulated in glycyrrhizic acid-hydrogel was equally effective in the treatment of Gardnerella vaginalis (FIG. 10).

1. A glycyrrhizic acid hydrogel comprising an imidazole or salt or solvate thereof for use in the prevention or treatment of a bacterial or fungal infection.

2. The hydrogel according to claim 1, for use in the prevention or treatment of a bacterial or fungal infection, wherein the hydrogel has a pH of between 2.5 and 5.

3. The hydrogel according to claim 1 or 2, for use in the prevention or treatment of a bacterial or fungal infection, wherein the hydrogel has a pH of between 3.5 and 4.5.

4. The hydrogel according to any one of claims 1 to 3, for use in the prevention or treatment of a bacterial or fungal infection, wherein the hydrogel has a pH of 4.

5. The hydrogel according to any one of claims 1 to 4, for use in the prevention or treatment of a bacterial or fungal infection, wherein the imidazole is miconazole, ketonazole or clotrimazole.

6. The hydrogel according to any one of claims 1 to 5, for use in the prevention or treatment of a bacterial or fungal infection, wherein the imidazole is miconazole.

7. The hydrogel according to any one of claims 1 to 6, for use in the prevention or treatment of a bacterial or fungal infection, wherein the concentration of the imidazole in the hydrogel is between 1.5 and 2.5 wt %.

8. The hydrogel according to any one of claims 1 to 7, for use in the prevention or treatment of a bacterial or fungal infection, wherein the concentration of glycyrrhizic acid is between 2.5 and 7.5 wt. %.

9. The hydrogel according to any one of claims 1 to 8, for use in the prevention or treatment of a bacterial or fungal infection, wherein the concentration of glycyrrhizic acid is between 4 and 6 wt. %.

10. The hydrogel according to any one of claims 1 to 9, for use in the prevention or treatment of a bacterial infection.

11. The hydrogel according to any one of claims 1 to 10, for use in the prevention or treatment of a bacterial infection, wherein the bacterial infection is an infection of Staphylococcus aureus or of Gardnerella vaginalis.

12. The hydrogel according to any one of claims 1 to 9, for use in the prevention or treatment of a fungal infection.

13. The hydrogel according to any one of claims 1 to 9, or to claim 12, for use in the prevention or treatment of a fungal infection, wherein the fungal infection is of Candida albicans or Candida glabrata.

14. A glycyrrhizic acid hydrogel characterized in that the hydrogel comprises an imidazole or salt or solvate thereof.

15. The hydrogel according to claim 14, wherein the hydrogel has a pH of between 2.5 and 5.

16. The hydrogel according to claim 14 or 15, wherein the hydrogel has a pH of between 3.5 and 4.5.

17. The hydrogel according to claim any one of claims 14 to 16, wherein the hydrogel has a pH of 4.

18. The hydrogel according to any one of claims 14 to 17, wherein the imidazole is miconazole, ketonazole or clotrimazole.

19. The hydrogel according to any one of claims 14 to 18, wherein the imidazole is miconazole.

20. The hydrogel according to any one of claims 14 to 19, wherein the concentration of the imidazole in the hydrogel is between 1.5 and 2.5 wt %.

21. The hydrogel according to any one of claims 14 to 20, wherein the concentration of the imidazole in the hydrogel is between 1.5 and 2.5 wt %.

22. The hydrogel according to any one of claims 14 to 21, wherein the concentration of glycyrrhizic acid is between 2.5 and 7.5 wt. %.

23. The hydrogel according to any one of claims 14 to 22, wherein the concentration of glycyrrhizic acid is between 4 and 6 wt. %.

24. The hydrogel according to any one of claims 14 to 23, wherein the concentration of glycyrrhizic acid is between 4 and 6 wt. %.

25. The hydrogel according to any one of claims 14 to 24, for use as a medicament.

26. A method of treating a bacterial or fungal infection comprising the step of administering an effective amount of hydrogel according to any one of claims 14 to 24.

27. A method of formulating an imidazole or salt or solvate thereof in a glycyrrhizic acid hydrogel, the method comprising the steps of:
a) preparing an aqueous solution of an imidazole,
b) heating the solution of a),
c) optionally adjusting the pH of the solution of a) or adjusting the pH of the heated solution of b) in order to solubilize the imidazole,
d) adding glycyrrhizic acid to the heated solution of b),
e) adjusting the pH of the solution of d) to a pH between 2.5 and 5,
f) cooling the solution of d) and allowing the formation of a hydrogel.

28. The method according to claim 27, wherein the imidazole is added in an amount to obtain a 2 wt. % concentration in the hydrogel.

29. The method according to claim 27 or 38, wherein the glycyrrhizic acid is added in an amount to obtain a 5 wt. % concentration in the hydrogel.

30. The method according to any one of claims 27 to 29, wherein in step a) or b) the imidazole is miconazole and the pH is adjusted to pH 7.

31. The method according to any one of claims 27 to 30, wherein in step a) or b) the imidazole is clotrimazole and the pH is adjusted to pH 2.

32. The method according to any one of claims 27 to 31, wherein in step e), the pH is adjusted to a pH between 3.5 and 4.5.

33. The method according to any one of claims 27 to 31, wherein in step e), the pH is adjusted to a pH of 4.
